(19) United States
(12) Patent Application Publication
(10) Pub. No.: US 2009/0004245 A1
(43) Pub. Date: Jan. 1, 2009

(54) Use of an iris simulated layer to allow aesthetic appearance drug loaded contact lens

(76) Inventors: Werhner C. Orilla, Anaheim, CA (US); Michael R. Robinson, Irvine, CA (US); James A. Burke, Santa Ana, CA (US)

Correspondence Address: Brent A. Johnson, Allergan, Inc. (T2-7H), 2525 Dupont Drive, Irvine, CA, 92612, US

(21) Appl. No.: 11/823,865

(22) Filed: Jun. 28, 2007

(51) Int. Cl. A61K 9/00

(52) U.S. Cl. 424/429

(57) ABSTRACT

A drug delivery device includes a contact lens along with a drug delivery zone disposed above an applied axis of the lens. A colored layer is disposed over the drug delivery zone in order to mark a color of the drug delivery zone.

The present invention is generally related to a delivery vehicle for dispensing therapeutic drugs and is more particularly directed to a biocompatible contact lens incorporating drugs in a manner enabling a sustained and effective dose release through the cornea and other surrounding tissue while at the same time providing a colored layer above or integrated into the contact lens in order that the eye appears more normal.

Drug loaded contact lenses have been described in the art, see for example U.S. Ser. No. 11/594,518 to Orilla, Burke, and Robinson. This application is to be incorporated herein in its entirety by this specific reference thereto.

Opaque and/or different colored drugs embedded in a contact lens body may be clearly seen by observers of the contact lens user and this appears un-natural.

On the other hand, colored contact lens have been disposed on the eye in an effort to alter the apparent color of the user's iris. See, for example U.S. Pat. Nos. 6,890,074 and 6,774,178. These patents are to be incorporated in their entirety into the present application by this specific reference thereto for the purpose of showing how colors may be incorporated into contact lens.

In addition, “soft contact lenses” have been utilized as a drug delivery vehicle. Soft contact lenses are formed from a highly porous plastic which can absorb water or other fluids and accordingly by saturating the lens with a fluid medication and inserting the lens into the eye a dosage of medication is provided in the eye by the lacrimal fluids. Unfortunately, these simple devices do not provide a proper control sustained release of the medication.

Other soft contact lenses have utilized a polymeric plastic in which a reservoir or medication is held, for example, U.S. Pat. Nos. 3,618,604 and 3,828,777. In these devices, the polymeric material is designed to control the release rate of the medication and thus provide a more uniform level of medication within the eye for extended periods of time.

In order to maintain clarity of vision, other medication delivering contact lenses have been developed with transparent central optic areas and peripheral areas with a drug embedded therein as in U.S. Pat. No. 3,786,812.

However, none of these devices have provided for zone delivery of a drug which may be necessary in instances such as lowering of intra ocular pressure (IOP) and at the same time provide for a natural appearance of the eye through the use of colored layers or pigment embedded into the contact lens to mask or color accommodate for coloration added by drug zones.

### SUMMARY OF THE INVENTION

A drug delivery device in accordance with the present invention generally includes a contact lens having an optical axis along with a drug delivery zone disposed about the optical axis. A color layer is disposed over the drug delivery zone in order to mask a color of the drug delivery zone.

More particularly, a plurality of discrete drug delivery zones may be asymmetrically disposed about the optical axis and the colored layer may include discrete color zone disposed over the corresponding drug delivery zones.

Preferably a combination of the drug zone color and the zone color matches a color of the colored layer outside of the color zones. In this manner, a uniform appearance may be provided for the iris.

In one embodiment, a single zone is used and in another embodiment a plurality of discrete drug delivery zones disposed in a spaced apart relationship within a central optical area surrounding the optic axis. The drug delivery zones may be disposed in an upper hemispherical region of the contact lens and further a weighted area in the contact lens may be provided for maintaining a presence of the contact lens upper hemispherical region in position with an upper hemispherical region of the user's eye.

The drug delivery zones may be of various shapes such as a circular, oblate, and in another embodiment of the present invention a second contact lens conforming to the contact lens may be provided with a drug delivery zone disposed between the contact lens and the second contact lens.

### BRIEF DESCRIPTION OF THE DRAWINGS

The present invention may be more clearly understood with reference to the following detailed description in connection, in which:

FIG. 1 is a plan view representing a contact lens with a single drug zone along with a colored simulated iris layer;

FIG. 2 shows the contact lens as shown in FIG. 1 in a assembled aesthetically appealing drug delivering contact lens;

FIG. 3 illustrates the assembly of a contact lens having a plurality of discrete delivery zones along with a color layer including discrete color zones for disposal over the corresponding drug delivery zones;

FIG. 4 is an assembled contact lens and color layer illustrating a generally uniform color only or pattern resulting from the combination of the color of drug zones and the color of the color zones;

FIG. 5 is a plan view of another embodiment of a contact lens in accordance with the present invention showing a plurality of discrete drug delivery zones asymmetrically disposed about an optical axis along with weighted areas;

FIG. 6 is a cross sectional view of the contact lens shown in FIG. 5 taken along the line 6-6;

FIG. 7 is a plan view of an alternative embodiment in accordance with the present invention also illustrating a plurality of discrete drug delivery zones asymmetrically disposed about an optical axis along with a weighted area; and

FIG. 8 is a cross sectional view of the contact lens shown in FIG. 3 taken along the line 7-7 and also showing a second contact lens conforming to a concave surface of the contact lens.

### DETAILED DESCRIPTION

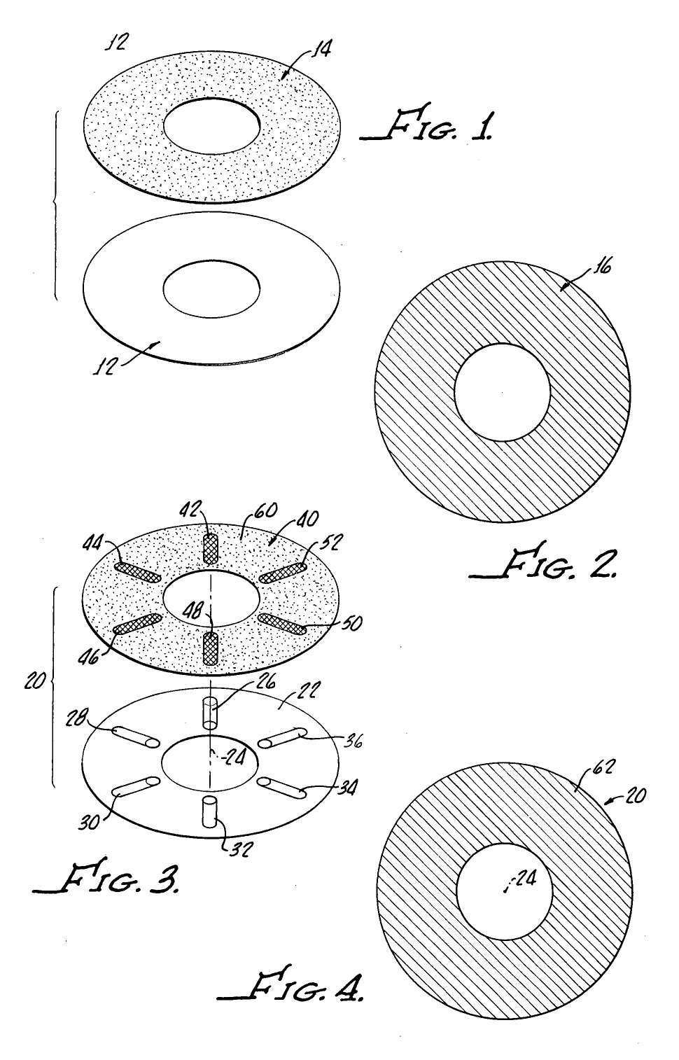

With reference to FIG. 1, there is shown a plan view representation of a drug loaded contact lens 12 being overlayed by a color layer 14 which results in an aesthetically appealing drug loaded contact lens 16 as shown in FIG. 2. The color layer 14 may be iris simulating.

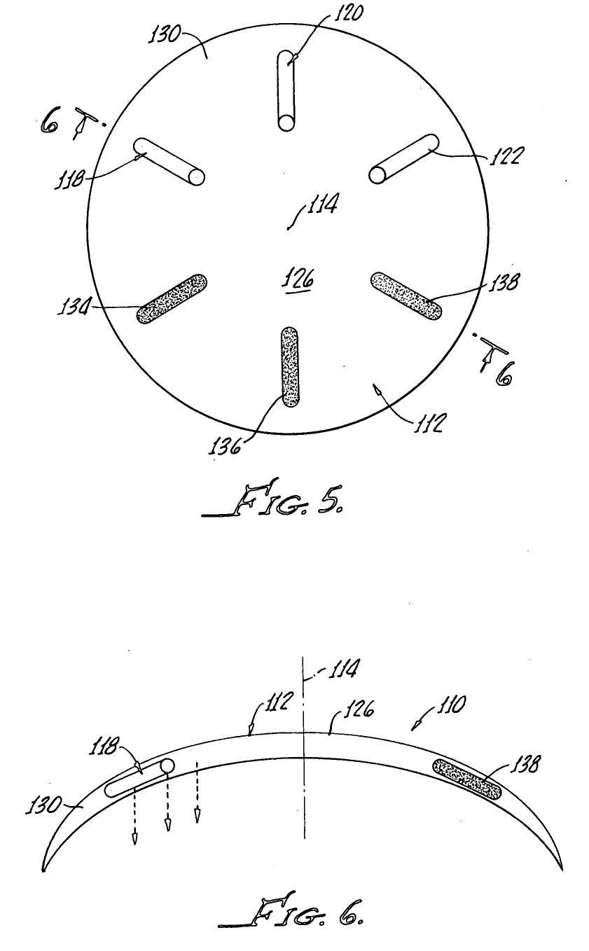

Turning to FIGS. 3 and 4, there is shown an alternative embodiment 20 in accordance with the present invention which generally includes a contact lens 22 having an optical axis 24. A plurality of discrete drug delivery zones 26, 28, 30, 32, 34, 36 are disposed asyemmetrically about the optical axis 24 as will be hereinafter described in greater detail. The drug may be brimonidine of a yellow color for treating interocular pressure.

A color layer 40 having discrete color zones 42, 44, 46, 48, 50, 52 is disposed over corresponding drug delivery zones 26, 28, 30, 32, 34, 36. The introduction of color and contact lenses is described in U.S. Pat. No. 6,890,075 which has been incorporated herewith for the purpose of describing the introduction of a plurality of pattern elements of different colors.

The colors are selected so that the combination of the drug zone are 26, 28, 30, 32, 34, 36 color and the zone colors 42, 44, 46, 48, 50, 52 match a background color in an area 60 of the color layer outside of the color zones 42, 44, 46, 48, 50, 52. This results in a file color area 62 as shown in FIG. 4, which may be uniform or match an iris-type coloration.

With reference to FIG. 5, there is shown a drug delivery device 110 in accordance with the present invention which generally includes a contact lens 112 having an optical axis 114. The lens 112 may cover the cornea and/or the peri-limbar area (not shown) and may be soft or hard utilizing silicone or any suitable commercial contact lens formulation.

A plurality of drug delivery zones 118, 120, 122 are asymmetrically disposed about the optical axis 114 generally in a spaced apart relationship with a central optical area 126. Any number of drug delivery zones 118, 120, 122 may be utilized with only three being shown in the embodiment 110 for illustrating the structure of the present invention.

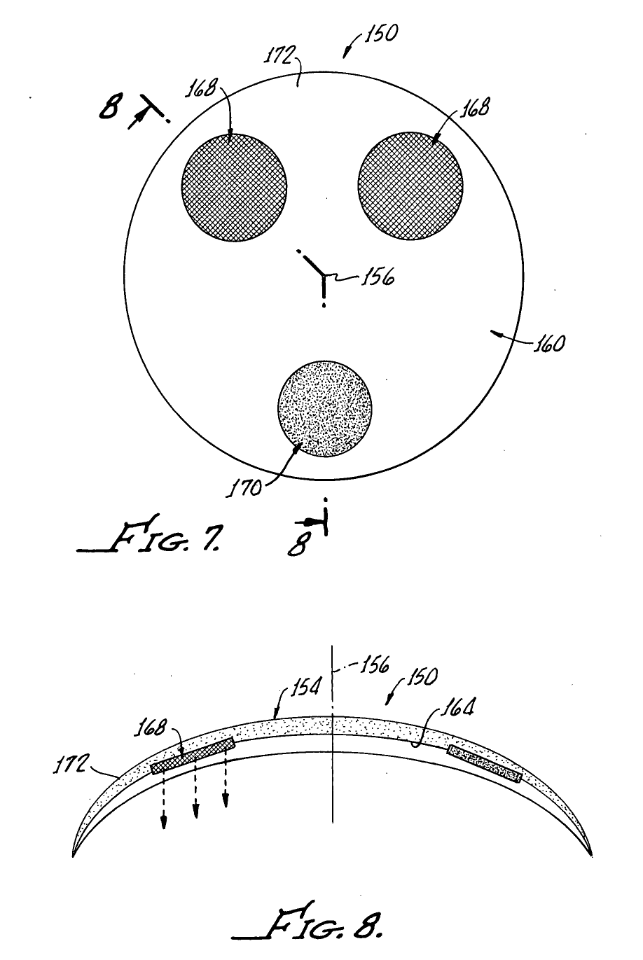

The zones 118, 120, 122 may be reservoirs for therapeutic drugs or the drugs may be incorporated into a PLA+PLGA matrix/microspheres zones allowing a controlled sustained release illustrated by the arrows 130 in FIG. 6. Such reservoir enable the use of higher concentrated active agents.

The zones 118, 120, 122 may be of any suitable shape and include, for example, any interocular pressure (IOP) lowering drug such as, for example, Brimo Tartrate+DDS PLA/PLGA rods.

The zones 118, 120, 122 are preferably disposed in a upper hemispherical region 130 for the purpose of avoiding the conjunctive, enhancing downward draining of the released drug, reducing wash and providing greater response of the eye to the drug.

In order to maintain a presence of the contact lens 112 upper hemispherical region 130 in position with an upper hemispherical region of a user's eye (not shown) weighted areas 134, 136, 138 are provided. It may be appreciated at any configuration of suitable weights including shape, size, and number may be utilized in the weighted areas or regions 134, 136, 138.

With reference to FIGS. 7 and 8, there is shown an alternative drug delivery device 150 in accordance with the present invention which includes a contact lens 154 having an optical axis 156 along with a second contact lens 160, as shown in FIG. 8, conforming to a concave surface 164 of a contact lens 154. A plurality of discrete drug delivery zones 168, 170 are asymmetrically disposed about the optical axis 156 and, as best shown in FIG. 8, between the contact lens 154 and the second contact lens 160. In this embodiment, the first contact lens 154 is an outer layer of non-hydrophilic material whereas the second contact lens 160 or inner layer, is a hydrophilic contact lens.

The zones 168, 170 may include Brimo Tartrate+DDS PLA/PLGA disc wafers which are sandwiched between the contact lens 154 and second contact lens 160. As with the embodiment 110, various shapes and numbers of drug delivery zones 168, 170 may be shown with two circular zones being shown for illustrative purposes only. The non-hydrophilic nature of the contact lens 154, or outer layer, prevents drug from leaking forward and the second contact lens 160, or inner layer, is hydrophilic which enables a drug to pass inwardly toward the eye (not shown).

As in the embodiment 110 a weighted area 170 maintains the orientation of the device 150 so that a presence of a contact lens upper hemispherical region 172 is in position with an upper hemispherical region of a user's eye (not shown).

A method for delivering a therapeutic and drug to a user's eye in accordance with the present invention includes providing a contact lens 110 having an optic axis 114 for insertion by a user into a user's cornea (not shown), disposing a plurality of drug delivery zones 118, 120, 122 asymmetrically about the optic axis 14 and instructing the user to install a contact lens 112 onto the user's cornea.

Further, a method for fabricating a drug delivery device 150 includes molding a contact lens 154 from a non-hydrophilic material, disposing a drug on a concave surface 164 of a contact lens 154 and thereafter molding a second contact lens 160 onto the concave surface 164 to sandwich the drug between the contact lens 154 and the second contact lens 160, the second contact lens being formed from a hydrophilic material.

Suitable non-hydrophilic materials include silicones such as unrestricted platinum fast-cure Nusil MED1-4213 and MED2-4123 and unrestricted Nusil low- and high-consistency elastomers with platinum cure systems. Alternatively, medical grade silicones commercially available may also be used as well as conventional hydrogel polymers.

Hydrophilic materials suitable for use in the present invention include: lower water content, non-ionic polymers (e.g. crofilcon); high water content, non-ionic polymers (e.g. lidofilcon; lower water content, ionic polymers (e.g. balafilcon); and high water content, ionic polymers (e.g. etafilcon A).

The color layers incorporated has been hereinbefore presented.

Although there has been hereinabove described a the use of an iris simulated layer to allow aesthetic appearance of a drug loaded contact lens in accordance with the present invention for the purpose of illustrating the manner in which the invention may be used to advantage, it should be appreciated that the invention is not limited thereto. That is, the present invention may suitably comprise, consist of, or consist essentially of the recited elements. Further, the invention illustratively disclosed herein suitably may be practiced in the absence of any element which is not specifically disclosed herein. Accordingly, any and all modifications, variations or equivalent arrangements which may occur to those skilled in the art, should be considered to be within the scope of the present invention as defined in the appended claims.

What is claimed is:

1. A drug delivery device comprising:
a contact lens having an optical axis;
a drug delivery zone disposed about said optical axis; and
a colored layer disposed over said drug delivery zone in order to mask a color of said drug delivery zone.

2. The device according to claim 1 further comprises a plurality of discrete drug delivery zone asymmetrically disposed about said optical axis.

3. The device according to claim 2 wherein said colored layer includes discrete color zones disposed over corresponding drug delivery zones.

4. The device according to claim 3 wherein a combination of the drug zone color and a corresponding zone color matches a color of the colored layer outside of the color zones.

5. The device according to claim 2 wherein said drug delivery zones are disposed in a spaced apart relationship within a central optical area surrounding said optical axis.

6. The device according to claim 5 wherein said drug delivery zones are disposed in an upper hemispherical region of said contact lens.

7. The device according to claim 6 further comprising a weighted area in said contact lens for maintaining a presence of the contact lens upper hemispherical region in position with an upper hemispherical region of a users' eye.

8. The device according to any one of claim 7 wherein said drug delivery zones are circular.

9. The device according to any one of claim 7 wherein said drug delivery zones are oblate.

10. The device according to claim 1 further comprising a second contact lens conforming to the contact lens and said drug delivery zone is disposed between said contact lens and said second contact lens.

11. The device according to claim 10 further comprises a plurality of discrete drug delivery zones asymmetrically disposed about said optical axis.

12. The device according to claim 11 wherein said drug delivery zones are disposed in a spaced apart relationship with a central optical area surrounding said optical axis.

13. The device according to claim 12 wherein said drug delivery zones are disposed in an upper hemispherical region of said contact lens.

14. The device according to claim 13 further comprising a weighted area in said contact lens for maintaining a presence of the contact lens upper hemispherical region in position with an upper hemispherical region of a users' eye.

15. The device according to any one of claim 11 wherein said drug delivery zones are circular.

16. The device according to any one of claim 11 wherein said drug delivery zones are oblate.

17. A drug delivery device comprising:
a contact lens having an optical axis;
a second contact lens conforming to said contact lens;
a drug delivery zone disposed about said optical axis and between the contact lens and the second contact lens. and
a colored layer disposed over said drug delivery zone in order to mask a color of said drug delivery zone.

18. The device according to claim 17 further comprises a plurality of discrete drug delivery zones asymmetrically disposed about said optical axis.

19. The device according to claim 18 wherein said colored layer includes discrete color zones disposed over corresponding drug delivery zones.

20. The device according to claim 19 wherein a combination of the drug zone color and the corresponding color zone matches a color of the colored layer outside of the color zones.

21. The device according to claim 13 wherein said drug delivery zones are disposed in a spaced apart relationship with a central optical area surrounding said optical axis.

22. The device according to claim 14 wherein said drug delivery zones are disposed in an upper hemispherical region of said contact lens.

23. The device according to claim 21 further comprising a weighted area in said contact lens for maintaining a presence of the contact lens upper hemispherical region in position with an upper hemispherical region of a users' eye.
